(19) United States
(12) Patent Application Publication
(10) Pub. No.: US 2023/0225854 A1
(43) Pub. Date: Jul. 20, 2023

(54) Intraocular non-perforating intra-scleral modular support system

(71) Applicant: Srinivas Dutt, Brooksville, FL (US)

(72) Inventor: Srinivas Dutt, Brooksville, FL (US)

(21) Appl. No.: 18/146,453

(22) Filed: Dec. 26, 2022

Related U.S. Application Data
(60) Provisional application No. 63/293,769, filed on Dec. 25, 2021.

(51) Int. Cl. A61F 2/16

(52) U.S. Cl. CPC A61F 2/16; A61F 2002/1686; A61F 2002/16903

(57) ABSTRACT

An intraocular support system is provided. This apparatus provides support when natural support structures are absent, diminished, or damaged. The apparatus may include a base that inserts into the internal scleral wall in the sulcus in an arched fashion that does not perforate the external scleral wall. It may be self-retaining with barb protrusions. It may include stabilizers that rest on the ciliary body. The base may include a modular junction that allows interchangeable extensions. The extensions may provide varied functions including intraocular lens support, capsular bag support, and accommodation. It may be applied in acute surgical conditions or in planned cases.

### CROSS-REFERENCE TO RELATED APPLICATION

This application claims the benefit of priority of U.S. provisional application No. 63/293,769, filed Dec. 25, 1921, the contents of which are herein incorporated by reference.

### BACKGROUND OF THE INVENTION

The present invention relates to an intraocular support system and, more particularly, to an intraocular support system that is non-perforating, intra-scleral, and modular.

Currently, there are situations where ocular surgeons support weakened or absent structures in the eye. One example includes significant loss of zonular integrity. Present treatment involves capsule tension rings or segments that are sutured to the sclera. Another example includes an intraocular lens which is subluxed and decentered out of visual axis. Present treatment involves suturing an intraocular lens to the sclera or performing an intraocular lens exchange. Another example involves no capsule support, aphakic, secondary intraocular lens, or intraocular lens exchange. Present treatments involve a sutured intraocular lens, externalized haptic with flange creation, or an iris sutured intraocular lens. Another situation includes pseudo-exfoliation with mild or no zonular loss. There is risk of progressive zonular weakness during or after surgery. Present treatment involves inserting a capsule tension ring.

Present techniques employed to provide intraocular support involve perforating the sclera, passing sutures through it, or pulling device parts out of it. These techniques are complex, have a steep learning curve, create complications related to scleral perforation and suture use, increase infection risk, cause late lens dislocation, are time intensive, and prolong operating time. Capsule Tension Rings may not be permanent solutions. Zonules can weaken over time leading to late capsule bag dislocation. It requires suturing or intraocular lens haptic externalization and flange creation to be secure. This is challenging with a steep learning curve. There are risks with scleral perforation and intraocular lens haptic manipulation. Sutures carry risk of infection, intraocular vitreous traction, and suture degradation.

Regarding accommodation, present techniques do not provide true accommodation, are limited, or may not provide adequate reconstitution of accommodation. Multifocal intraocular lens can provide multiple images and varied focal length giving pseudo-accommodation. It can lead to visual distortions and halos that are debilitating for some patients. Laser corneal treatments provide pseudo-accommodation but have variable results and visual side effects. Scleral relaxing incisions purport to increase accommodation but has not shown significant efficacy.

As can be seen, there is a need for an improved intraocular support apparatus. This present invention avoids complications of present techniques with no scleral perforation and no suture use. This device avoids contortion forces on the globe, loss of globe integrity, potential for scleral perforation infections, and potential for suture degradation and tissue erosion over time. It involves a fast maneuver that saves operating time. Its ease of insertion is within existing comfort levels of ocular surgeons without the need to learn new skills.

### SUMMARY OF THE INVENTION

In one aspect of the present invention, an intraocular support apparatus comprises: a base platform and extensions. The base platform may include insertion arms, stalk, stabilizer, and a modular junction. The extensions may be varied to provide different functions and include a junction to attach to the base.

In another aspect of the present invention, an intraocular apparatus gives structural support when intrinsic biologic support is insufficient or missing. It can be inserted into the internal sclera within the sulcus without full thickness perforation of the scleral wall. It can be self-retaining using curved tips with barb protrusions. It can enter the internal sclera in an arched fashion that allows increased tissue engagement without outer scleral wall perforation. No sutures are involved avoiding infection and degradation risk. It involves an efficient process that saves operating time. The device can be self-stabilizing with various embodiments of stabilizers in the sulcus. The device can be multifunctional using modular interchangeable extensions. It can also provide a modality for accommodation. The device may consist of biocompatible materials. This device improves safety and efficacy in ophthalmic anterior segment surgery.

These and other features, aspects, and advantages of the present invention will become better understood with reference to the following drawings, descriptions, and claims.

### BRIEF DESCRIPTION OF THE DRAWINGS

FIG. 1 is a perspective of the present invention illustrating the base platform;

FIG. 2 is a perspective of the present invention illustrating an example of an extension;

FIG. 3 is a section view of the present invention, illustrating the placement of the arched insertion tips into the sclera without perforation of the outer wall.

### DETAILED DESCRIPTION OF THE INVENTION

The following is a detailed description of the best currently contemplated modes of carrying out exemplary embodiments of the invention. The description is not to be taken in a limiting sense, but is made merely for the purpose of illustrating the general principles of the invention.

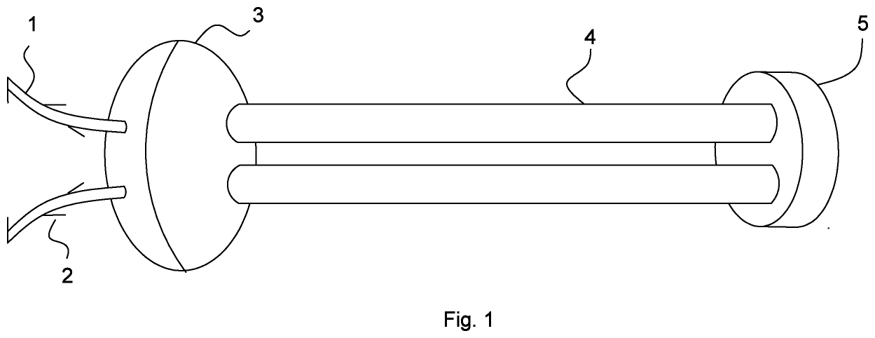

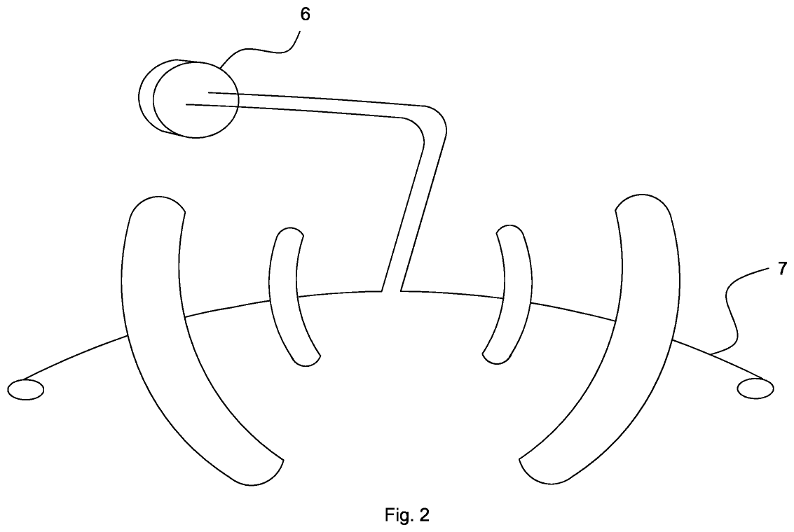

The present apparatus includes a base platform and modular extensions as referenced in FIG. 1 and FIG. 2. In FIG. 1 the base platform involves a stalk 4 with three components: Insertion arms 1, a stabilizer 3, and a modular junction 5.

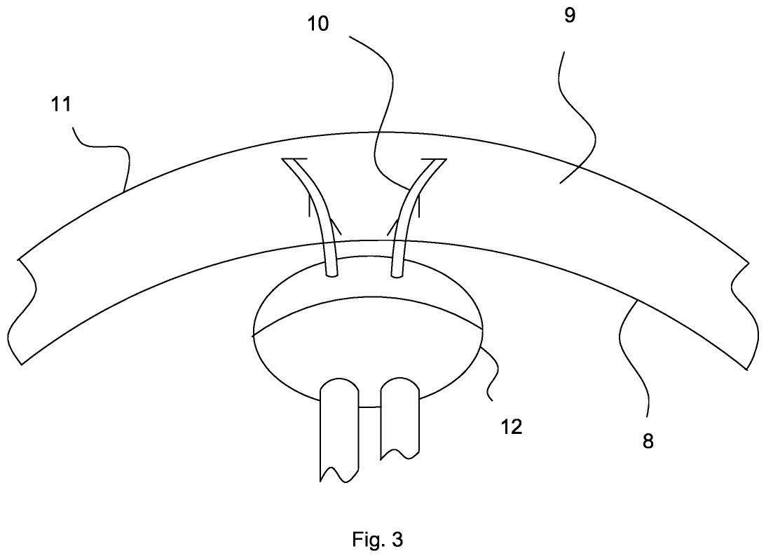

Insertion arms include barb protrusions 2 that prevent retraction. Multiple barbs assist retention unlike single flange or single knot with sutures or haptics. As referenced in FIG. 3, the distal sharp arched tips 10 are curved outward to allow internal scleral wall 8 entry and internal tissue 9 engagement without perforating the outer scleral wall 11. This results in greater insertion length with curved entry relative to straight entry with sutures. Additional arms can be employed as needed for increased support force. T base platform can be attached to the internal scleral wall with forceps or specialized equipment to inject it into the sulcus above the ciliary body from any angle. This apparatus allows ease and speed of insertion by following the iris plane until scleral engagement. Multiple devices can be placed in multiple quadrants for additional support. This device avoids sutures, intraocular lens haptic externalization, scleral perforation and their potential complications.

The insert rests in the sulcus between the ciliary body and iris. The stabilizer 12 in FIG. 3 rests in the sulcus on the ciliary body. Stabilizers along with the insertion arms prevent any unwanted movement in the x, y, or z axis. The stabilizers could be thick or thin, loops, or solid shapes. A sphere or ellipsoid, separately or combined, could provide gentle stability. Rounded contour protects adjacent structures.

The modular junction 5 in FIG. 1 allows interchangeable extensions to attach into the insertion base at this junction. As referenced in FIG. 2 the extension 7 has a junction 6 to attach to the base. Placement of extensions into the junction is adjustable allowing titration of force. Modular extensions can create a multifunctional system that can include zonule replacement, capsule support, partial or complete intraocular lens support, hook for dislocated intraocular lens, and flexibility for future options. Variations of these examples could also exist. A smaller size support could be employed if only a single sector of the capsule needs support. Small support bars could be utilized for added support with larger implants if needed. Loops could be used to fixate a dislocated intraocular lens. Lens support alone could be another variation.

The device includes an accommodation function. This device rests in the sulcus on the ciliary body. As the ciliary body contracts and relaxes, some force is transmitted to the stabilizers. This force can be amplified by modifying the width and shape of the stabilizers to maximize transmission of ciliary muscle contraction force. This force is transmitted through an extension that rests under the intraocular lens, acting as a lever, further amplifying muscle force and providing accommodation. A modified version with inferior lens support that could slip under an existing intraocular lens could provide accommodation to existing pseudophakic patients. An ellipsoid stabilizer could smoothly amplify reactions from the ciliary body. An extended stabilizer could maximize amplification of existing accommodative function. This device can bypass zonular laxity and lens stiffness components of accommodation loss. It enhances natural accommodation. It can be used with any posterior chamber intraocular lens. It can be used with a previously implanted posterior chamber intraocular lens. No visual distortion or halos would occur.

This device can be manufactured with materials that are malleable and biocompatible. Implant material would be flexible with memory. This allows for long term placement and allows constriction in order to insert through a small corneal opening and expands once intraocular. Different sizes, styles, and shapes of the various components can be used for different functions. The above can also be modified and reconfigured to create the same or similar functions.

While exemplary embodiments have been described and illustrated, these should not be construed as limitations on the scope of what is claimed or on what may be claimed. Various modifications and enhancements can be made without departing from the spirit and scope of the invention.

What is claimed is:

1. An intraocular support apparatus comprising: a base platform and modular extensions.

2. The intraocular support apparatus of claim 1, further comprising a base platform wherein in it involves a stalk with 3 components: Insertion arms, a stabilizer, and a modular junction.

3. The intraocular support apparatus of claim 2, further comprising insertion arms wherein they are arched allowing increased scleral entry without perforation of the outer scleral wall.

4. The intraocular support apparatus of claim 3, further comprising insertion arms wherein there are barb protrusions supporting retention in tissue.

5. The intraocular support apparatus of claim 2, further comprising a stabilizer wherein it rests in the sulcus on the ciliary body promoting stability.

6. The intraocular support apparatus of claim 2, further comprising a stabilizer wherein it rests in the sulcus on the ciliary body providing accommodation.

7. The intraocular support apparatus of claim 2, further comprising a modular junction wherein it provides attachment of extensions.

8. The intraocular support apparatus of claim 1, further comprising extensions wherein there are varied shapes, styles, and functions.

9. A method of attaching the apparatus of claim 3 intraocularly to the globe wherein the arched insertion arms enter the internal scleral wall, traverse in a curved fashion allowing greater tissue penetration, and avoid perforation of the outer scleral wall.

10. A method of attaching the apparatus of claim 4 intraocularly to the globe, further comprising barb protrusions on the arched arms that prevent retraction.

11. A method of stabilizing the apparatus of claim 6 intraocularly wherein the insertion arms and stabilizer prevent movement along a dimensional axis.

12. A method of achieving accommodation wherein stabilizers of claim 6 that rest in the sulcus on the ciliary body amplify and transmit accommodative force to an intraocular lens.

13. A method of attaching modular extensions of claim 7 wherein extensions of varied functions attach to the base at a modular junction.

14. A method of utilizing extensions of claim 8 wherein they are varied in but not limited to size, shape, materials, and functions.
